(19) United States
(12) Patent Application Publication
(10) Pub. No.: US 2009/0148850 A1
(43) Pub. Date: Jun. 11, 2009

(54) Methods for identifying modulators of P2RY14

(76) Inventors: Stacia Kargman, Montreal (CA); Martine Hamel, Laval (CA); Joseph A. Mancini, Kirkland (CA); Christopher Bayly, Beaconsfield (CA)

Correspondence Address: MERCK AND CO., INC, P O BOX 2000, RAHWAY, NJ, 07065-0907, US

(21) Appl. No.: 12/286,566

(22) Filed: Oct. 1, 2008

Related U.S. Application Data
(60) Provisional application No. 60/997,614, filed on Oct. 4, 2007.
(60) Provisional application No. 61/133,701, filed on Jul. 1, 2008.

(51) Int. Cl. C12Q 1/68

(52) U.S. Cl. 435/6

(57) ABSTRACT

Methods for identifying modulators of P2RY14 are described. The methods are particularly useful for identifying agents that are useful for treating metabolic syndrome.

### BACKGROUND OF THE INVENTION

(1) Field of the Invention

The invention relates to methods for identifying modulators of P2RY14, in particular, modulators that are P2RY14 antagonists. The methods are particularly useful for identifying agents that are useful for treating metabolic syndrome.

(2) Description of Related Art

Metabolic Syndrome is a disorder that is a combination of medical disorders that increase one's risk for cardiovascular disease, stroke, and diabetes and includes obesity, dyslipidaemia, and hyperglycemia. Metabolic syndrome, which is also known as (metabolic) syndrome X, insulin resistance syndrome, Reaven's syndrome, and CHAOS (Australia), has increased to epidemic proportions worldwide. The pathophysiology of this syndrome is attributed to central distributed obesity, decreased high density lipoprotein, elevated triglycerides, elevated blood pressure and hyperglycemia. People suffering from Metabolic Syndrome are at increased risk of type II diabetes, coronary heart disease, and other diseases related to plaque accumulation in artery walls (e.g., stroke and peripheral vascular disease). In two prospective European studies, Metabolic Syndrome was a predictor of increased cardiovascular disease and mortality (Isomaa et al., Diabetes Care 24: 683-689 (2001); Lakka et al., JAMA 288: 2709-2716 (2002)).

The most significant underlying cause of Metabolic Syndrome appears to be obesity. The genetic factors that also contribute to Metabolic Syndrome are not yet understood. Consequently, there is a need to identify genes that contribute to the development of Metabolic Syndrome. There is also a need for methods that permit the identification of chemical agents that modulate the activity of these genes or modulate the activity of the products (e.g., proteins) encoded by these genes. Such chemical agents may be useful, for example, as drugs to prevent Metabolic Syndrome or to ameliorate at least one symptom of Metabolic Syndrome.

International Patent Application No. WO03076945 discloses a human GPR105 and stated it was useful in assays for identification of compounds useful for the treatment or prevention of genitor-urinary diseases, disorders of the nervous system, hematology diseases, cardiovascular diseases, gastro-intestinal diseases, metabolic diseases and cancer. U.S. Published Patent Application No. 20070092913 disclosed that expression of GPR105 protein is correlated with weight gain and development of type II diabetes. Further, the application demonstrated that antisense inhibition of GPR105 expression in mice reduced the rate at which the mice gain weight in response to a high fat diet. The mice also have lower levels of insulin, suggesting a decreased level of insulin resistance in these mice. Accordingly, GPR105 is a target for drugs that prevent diabetes, obesity or Metabolic Syndrome, or that ameliorate at least one symptom of Metabolic Syndrome.

GPR105 is a member of the pyrimidinergic (P2RY) G-protein coupled receptor (GPCR) gene family, which includes 8 human members, P2RY1, 2, 4, 6, 11, 12, 13 and 14. Currently, GPR105 is referred to as P2RY14. The sequence identity among the P2RY family members ranges from 13-51% with P2RY14 being most similar to P2RY12 and P2RY13, with 44% and 42% sequence identity, respectively. P2RY14 is ubiquitously expressed in human tissues with the highest expression seen in adipose tissue and stomach. In mouse, P2RY14 is also ubiquitously expressed with the highest expression demonstrated in adrenal gland and spleen, and moderate expression in adipose tissue. P2RY14 is alleged to signal via Gi and the consequent inhibition of cAMP (Scrivens and Dickenson, Brit. J. Pharmacol. 146: 435-444 (2005)) or via Gq and the consequent increase in calcium flux (Skelton et. al., J. Immunol. 171: 1941-1949 (2003)). Although signaling has been reported to be Pertussis toxin sensitive (indicating Gi/o signaling) (Chambers et. al., J. Biol. Chem. 275: 10767-10771 (2000); Skelton et al., J. Immunol. 171: 1941-1949 (2003)), compelling evidence for P2RY14 agonist-induced inhibition of cAMP is limited.

Chambers et al. (J. Biol. Chem. 275: 10767-10771 (2000)) reported that nucleotide sugars (UDP-glucose, UDP-galactose, UDP-glucuronic acid and UDP-N-acetylglucosamine) were able to activate P2RY14 in both yeast and mammalian cells. In contrast, other sugar-nucleotides such as ADP-glucose, naturally occurring nucleotides (UMP, UDP, UTP) and nucleosides were unable to activate P2RY14. Ames et al. (U.S. Pat. No. 6,238,873) discloses an assay for modulators of GPR105 under conditions that permit binding to GPR105 in the presence of a labeled or unlabeled UDP sugar selected from the group consisting of UDP-glucose, UDP-galactose, UDP-glucoronic acid, and UDP-N-acetyl glucosamine. Fricks et al. (The FASEB J. 21: 568.14 (2007) disclosed that UDP was a competitive antagonist for the human P2YR14 receptor but a full agonist for the rat P2YR14 receptor.

The inventors have discovered that UMP, UDP, and UTP each act as an agonist of P2RY14 activity. This discovery has led to the design of new assays for identifying novel P2RY14 antagonists.

### BRIEF SUMMARY OF THE INVENTION

The present invention provides methods for identifying modulators or antagonists or agonists of P2RY14 activity. The methods include functional and binding assays for identifying the modulators of P2RY14 activity. The methods are useful for identifying agents that are useful for treating metabolic syndrome. The methods are particularly useful for identifying agents that are useful for treating diabetes, cardiovascular disease, stroke, obesity, dyslipidaemia, and hyperglycemia.

In one aspect, provided is a method or functional assay for identifying agents that modulate pyrimidinergic receptor P2Y, G-protein coupled, 14 (P2RY14) protein activity comprising contacting a cell expressing on the surface thereof the P2RY14 protein, the P2RY14 protein being associated with a second component capable of providing a detectable signal in response to the activation of P2RY14 protein, with an agent to be screened in the presence of a labeled or unlabeled nucleotide selected from the group consisting of inosine diphosphate, UMP, UDP, and UTP; and determining whether the agent modulates the P2RY14 protein activity by measuring the level of a signal generated from the interaction of the agent with the P2RY14 protein, wherein a change in the signal from that obtained in the absence of the agent identifies the agent as a modulator of the P2RY14 protein activity. When the signal is decreased, the agent is an antagonist of the P2RY14 protein, and when the signal is increased, the agent is an agonist of the P2RY14 protein.

In another aspect, provided is a method or functional assay for identifying agents that are antagonists of P2RY14 protein activity comprising contacting a cell expressing on the surface thereof the P2RY14 protein, the P2RY14 protein being associated with a second component capable of providing a detectable signal in response to the activation of P2RY14 protein, with an agent to be screened in the presence of a labeled or unlabeled nucleotide selected from the group consisting of inosine diphosphate, UMP, UDP, and UTP; and determining whether the agent antagonizes the P2RY14 protein activity by measuring the level of a signal generated from the interaction of the agent with the P2RY14 protein, wherein a decrease in the signal from that obtained in the absence of the agent identifies the agent as antagonist of the P2RY14 protein activity.

In another aspect, provided is a method or functional assay for identifying agents that are agonists of P2RY14 protein activity comprising contacting a cell expressing on the surface thereof the P2RY14 protein, the P2RY14 protein being associated with a second component capable of providing a detectable signal in response to the activation of P2RY14 protein, with an agent to be screened in the presence of a labeled or unlabeled nucleotide selected from the group consisting of inosine diphosphate, UMP, UDP, and UTP; and determining whether the agent agonizes the P2RY14 protein activity by measuring the level of a signal generated from the interaction of the agent with the P2RY14 protein, wherein an increase in the signal from that obtained in the absence of the agent identifies the agent as an agonist of the P2RY14 protein activity.

In a further still aspect, provided is a method for identifying agents that competitively inhibit binding of UTP/UDP to a P2RY14 protein, comprising measuring binding of a labeled or unlabeled UTP/UDP to cells having the P2RY14 protein on the surface thereof, or to cell membranes containing the P2RY14 protein, in the presence of the agent under conditions to permit binding of the UTP/UDP to the P2RY14 protein in the absence of the agent; and determining the amount of the UTP/UDP bound to the P2RY14 protein, wherein an agent that causes a reduction of binding of the UTP/UDP to the P2RY14 protein competitively inhibits the binding of the UTP/UDP to the P2RY14 protein. Agent that competitively inhibits binding of the UTP/UDP to the P2RY14 protein can be a modulator of P2RY14 activity. The aforementioned functional assays for identifying modulators of P2RY14 protein can be used determine whether the agent that competitively binds the P2RY14 protein is a modulator of P2RY14 activity.

In a further still aspect, provided is a method for identifying agents that bind a pyrimidinergic receptor P2Y, G-protein coupled, 14 (P2RY14) protein and modulate P2RY14 activity comprising (a) contacting a cell expressing on the surface thereof the P2RY14 protein, the P2RY14 protein being associated with a second component capable of providing a detectable signal in response to the activation of P2RY14 protein, with an agent to be screened in the presence of a labeled or unlabeled nucleotide selected from the group consisting of inosine diphosphate, UMP, UDP, and UTP; (b) determining whether the agent modulates the P2RY14 protein activity by measuring the level of a signal generated from the interaction of the agent with the P2RY14 protein, wherein a change in the signal from that obtained in the absence of the agent identifies the agent as a modulator of the P2RY14 protein activity; (c) contacting cells having the P2RY14 protein on the surface thereof, or cell membranes containing the P2RY14 protein, with the agent from step (b) in the presence of labeled or unlabeled UTP/UDP under conditions to permit binding of the UTP/UDP to the P2RY14 protein in the absence of the agent; (d) measuring binding of a labeled or unlabeled UTP/UDP to the cells or membranes; and (e) determining the amount of the UTP/UDP bound to the P2RY14 protein, wherein an agent that causes a reduction of binding of the UTP/UDP to the P2RY14 protein binds the P2RY14 protein and modulates P2RY14 protein activity.

In a further still aspect, provided is a method for identifying agents that bind a pyrimidinergic receptor P2Y, G-protein coupled, 14 (P2RY14) protein and modulate P2RY14 activity comprising (a) contacting cells having the P2RY14 protein on the surface thereof, or cell membranes containing the P2RY14 protein, with an agent to be screened in the presence of labeled or unlabeled UTP/UDP under conditions to permit binding of the UTP/UDP to the P2RY14 protein in the absence of the agent; (b) measuring binding of a labeled or unlabeled UTP/UDP to the cells or membranes; (c) determining the amount of the UTP/UDP bound to the P2RY14 protein, wherein an agent that causes a reduction of binding of the UTP/UDP to the P2RY14 protein binds the P2RY14 protein; (d) contacting a cell expressing on the surface thereof the P2RY14 protein, the P2RY14 protein being associated with a second component capable of providing a detectable signal in response to the activation of P2RY14 protein, with the agent from step (c) in the presence of a labeled or unlabeled nucleotide selected from the group consisting of inosine diphosphate, UMP, UDP, and UTP; (e) determining whether the agent inhibits the P2RY14 protein activity by measuring the level of a signal generated from the interaction of the agent with the P2RY14 protein, wherein a change in the signal from that obtained in the absence of the agent identifies the agent as a modulator of the P2RY14 protein activity.

In particular embodiments of the above, the cell is a mammalian cell, which in particular embodiments can be a HEK293 cell. In further still embodiments, the P2RY14 protein has an amino acid sequence selected from the group consisting of SEQ ID NO: 1, SEQ ID NO:2, SEQ ID NO:3, SEQ ID NO:4; SEQ ID NO:5, SEQ ID NO:6, and SEQ ID NO:7.

In any one of the first two embodiments, the second component can comprise a chimeric Gqi5 protein and the detectable signal can comprise calcium signaling.

As used herein, the term “agent” encompasses any biological or chemical molecule, or chemical element, or a combination of chemical molecules and/or chemical elements. For example, the term “agent” encompasses proteins (comprising at least 100 covalently linked amino acid units) and peptides (comprising from 2 to 99 covalently linked amino acid units). Again, by way of example, the term “agent” encompasses molecules that mimic the ligands for a P2RY14 protein. An agent that mimics a ligand of P2RY14 that stimulates P2RY14 is an agonist of P2RY14 activity and an agent that mimics a ligand that inhibits P2RY14 activity is an antagonist of P2RY14 activity.

As used herein, the term “P2RY14 protein” refers to a type of G-protein coupled receptor. The natural ligand for P2RY14 protein is not known, but UDP-hexoses (e.g., UDP-glucose) bind the receptor with high affinities (10-500 nM). UDP-glucose is an activated form of glucose used for glycogen synthesis. As shown herein, nucleotides UMP, UDP, and UTP also bind the receptor with high affinities. The biological function of P2RY14 is not known, but it may have a role in cellular chemotaxis and inflammation. Human body atlas data shows that P2RY14 is predominantly expressed in the intestines and subcutaneous white adipose tissue. Mouse data shows highest expression of P2RY14 in spleen and pancreas, with only average expression in brain.

Some P2RY14 proteins useful in the practice of the present invention are at least 79% identical (e.g., at least 80% identical, or at least 90% identical, or at least 95% identical, or at least 99% identical) to the human P2RY14 protein having the amino acid sequence set forth in SEQ ID NO: 1 (GenBank Accession No. XP_{—}055694), and encoded by the transcript having GenBank Accession No. NM_{—}014879. Other P2RY14 proteins include, but are not limited to, those having the amino acid sequence shown in GenBank Accession Nos. XP_{—}001107208 (Rhesus monkey, SEQ ID NO:2), NP_{—}598261 (rat, SEQ ID NO:3), XP_{—}542838 (dog, SEQ ID NO:4), NP_{—}573463 (mouse, SEQ ID NO:5), XP_{—}001145005 (chimpanzee, SEQ ID NO:6), and XP_{—}422841 (chicken, SEQ ID NO:7).

The term “percent identity” or “percent identical” when used in connection with amino acid sequence relatedness between P2RY14 proteins, is defined as the percentage of amino acid residues in a first P2RY14 protein sequence that are identical with a second P2RY14 protein sequence (such as the P2RY14 amino acid sequence of SEQ ID NO: 1), after aligning the first and second P2RY14 sequences to achieve the maximum percent identity. For example, percentage identity between two protein sequences can be determined by pairwise comparison of the two sequences using the b12seq interface at the Web site of the National Center for Biotechnology Information (NCBI), U.S. National Library of Medicine, 8600 Rockville Pike, Bethesda, Md. 20894, U.S.A. The b12seq interface permits sequence alignment using the BLAST tool described by Tatiana et al., “Blast 2 Sequences—A New Tool for Comparing Protein and Nucleotide Sequences,” FEMS Microbiol. Lett. 174:247-250 (1999). The following alignment parameters are used: Matrix=BLOSUM62; Gap open penalty=11; Gap extension penalty=1; Gap x_dropff=50; Expect=10.0; Word size=3; and Filter=off.

As used herein, the term “biological activity” refers to an effect of a P2RY14 protein on a biological process in a living cell, living tissue, living organ and/or living organism. Examples of biological processes include biochemical pathways, concentration of one or more chemical compounds within a living cell, physiological processes that contribute to the internal homeostasis of a living organism, developmental processes that contribute to the normal physical development of a living organism, and acute or chronic diseases.

Modulation of the biological activity of a P2RY14 protein encompasses any change in a biological activity of a P2RY14 protein. For example, the change in biological activity can be a decrease in the biological activity of a P2RY14 protein (e.g., complete, or substantially complete, inhibition of a biological activity of a P2RY14 protein). Again by way of example, the change in biological activity can be a reduction in the rate of a biological activity of a P2RY14 protein. Again by way of example, the change in biological activity can be an increase in the activity of a P2RY14 protein.

### BRIEF DESCRIPTION OF THE DRAWINGS

FIG. 1A shows the results of a FLIPR assay measuring the agonist effect of UDP-glucose, UDP-galactose, UDP-glucuronic acid, and UDP-N-acetylglucosamine on human P2RY14 activity.

FIG. 1B shows the results of a FLIPR assay measuring the agonist effect of UDP-glucose, UDP-galactose, UDP-glucuronic acid, and UDP-N-acetylglucosamine on simian (chimpanzee) P2RY14 activity.

FIG. 1C shows the results of a FLIPR assay measuring the agonist effect of UDP-glucose, UDP-galactose, UDP-glucuronic acid, and UDP-N-acetylglucosamine on mouse P2RY14 activity.

FIG. 1D shows the results of a FLIPR assay measuring the agonist effect of UMP, UDP, and UMP compared to UDP-glucose on human P2RY14 activity.

FIG. 1E shows the results of FIG. 1D with data cut off for nucleotides at 1 μM.

FIG. 1F shows the results of a FLIPR assay measuring the agonist effect of UMP, UDP, and UMP compared to UDP-glucose on simian (chimpanzee) P2RY14 activity.

FIG. 1G shows the results of a FLIPR assay measuring the agonist effect of UMP, UDP, and UMP compared to UDP-glucose on mouse P2RY14 activity.

FIG. 2A shows the results of a CellKey™ assay measuring the agonist effect of UDP-glucose, UDP-galactose, UDP-glucuronic acid, and UDP-N-acetylglucosamine on human P2RY14 activity wherein the results are normalized to UDP-glucose set at 1.0.

FIG. 2B shows the results of a CellKey™ assay measuring the agonist effect of UDP-glucose, UDP-galactose, UDP-glucuronic acid, and UDP-N-acetylglucosamine on simian (chimpanzee) P2RY14 activity wherein the results are normalized to UDP-glucose set at 1.0.

FIG. 2C shows the results of a CellKey™ assay measuring the agonist effect of UDP-glucose, UDP-galactose, UDP-glucuronic acid, and UDP-N-acetylglucosamine on mouse P2RY14 activity wherein the results are normalized to UDP-glucose set at 1.0.

FIG. 2D shows the results of a CellKey™ assay measuring the agonist effect of UMP, UDP, and UMP compared to UDP-glucose on human P2RY14 activity wherein the results are normalized to UDP-glucose set at 1.0.

FIG. 2E shows the results of FIG. 2D with data cut off for nucleotides at 1 μM.

FIG. 2F shows the results of a CellKey™ assay measuring the agonist effect of UMP, UDP, and UMP compared to UDP-glucose on simian (chimpanzee) P2RY14 activity wherein the results are normalized to UDP-glucose set at 1.0.

FIG. 2G shows the results of a CellKey™ assay measuring the agonist effect of UMP, UDP, and UMP compared to UDP-glucose on mouse P2RY14 activity wherein the results are normalized to UDP-glucose set at 1.0.

FIG. 3 compares the specificity of UMP, UDP, and UTP agonist activity.

FIG. 4A shows the binding specificity of [³H]-UDP-glucose to simian (chimpanzee) P2RY14.

FIG. 4B shows the binding specificity of [³H]-UMP to simian (chimpanzee) P2RY14.

FIG. 4C shows the binding specificity of [³H]-UTP/[³H]-UDP to simian (chimpanzee) P2RY14.

FIG. 5A shows the results of a competition binding assay between [³H]-UTP/[³H]-UDP and UDP-glucose, UMP, UDP, and compound A to simian (chimpanzee) P2RY14.

FIG. 5B FIG. 5A shows the results of a competition binding assay between [³H]-UTP/[³H]-UDP and UDP-glucose, UMP, UDP, and compound A to human P2RY14.

FIG. 6A shows that compound A identified in the FLIPR assay inhibits or antagonizes P2RY14 activity.

FIG. 7A shows the HPLC analysis of [³H]-UTP stability in the P2RY14 membrane filtration binding assay at time T=0.

FIG. 7B shows the HPLC analysis of [³H]-UTP stability in the P2RY14 membrane filtration binding assay at time T=1 minute.

FIG. 7C shows the HPLC analysis of [³H]-UTP stability in the P2RY14 membrane filtration binding assay at time T=60.

FIG. 7D shows the time course of [³H]-UTP conversion in the P2RY14 membrane filtration binding assay.

FIGS. 8A and 8B show the HPLC analysis of [³H]-UTP stability after the 10 minute cell-based FLIPR assay.

### DETAILED DESCRIPTION OF THE INVENTION

The present invention provides a method for identifying modulators of P2RY14 activity in an assay that uses UMP, UDP, or UTP as a P2RY14 agonist. In practice of the invention, a cell culture is provided in which the cells express P2RY14 on the cell surface. The cells are contacted with an agent in the presence of UMP, UDP, or UTP. It is then determined whether the agent modulates the biological activity of a P2RY14 protein in the cell. The method can be used in assays to screen agents to identify agents that are antagonists of P2RY14. The method can also be used to screen agents to identify agents that are agonists of P2RY14. The method is useful for identifying agents that are useful for treating metabolic syndrome. The method is particularly useful for identifying agents that are useful for treating diabetes, cardiovascular disease, stroke, obesity, dyslipidaemia, and hyperglycemia.

We have discovered that in addition to UDP-glucose, inosine diphosphate, UMP, UDP, and UTP uncoupled to glucose also activated P2RY14 in HEK cells expressing human, Chimpanzee, or mouse P2RY14 in combination with Gqi5. Stable HEK clonal cell lines expressing P2RY14 and the chimeric G protein, Gqi5 had been constructed. The chimeric Gqi5 forces the coupling of P2RY14 through the Gq pathway and allows for monitoring of agonist-induced calcium signaling using a calcium binding fluorescent dye and a FLIPR (fluorometric imaging plate reader). Using these clonal cell lines in the presence of UDP-glucose, UDP-galactose, UDP-glucuronic acid, or UDP-N-acetylglucosamine, the results of Chambers et. al. (J. Biol. Chem. 275: 10767-10771 (2000)) were confirmed.

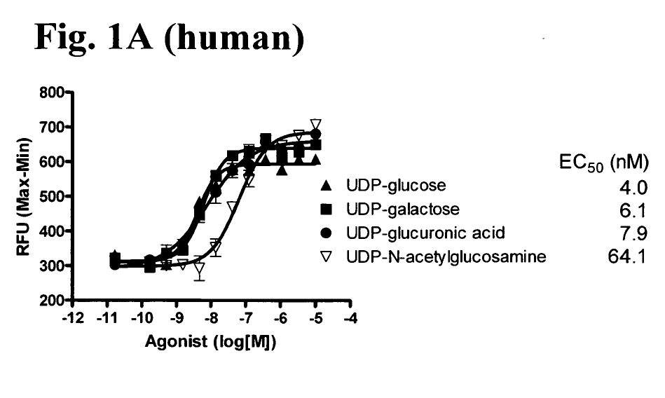

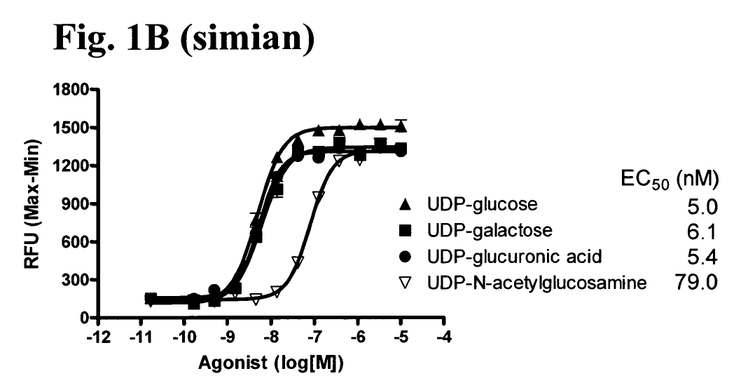

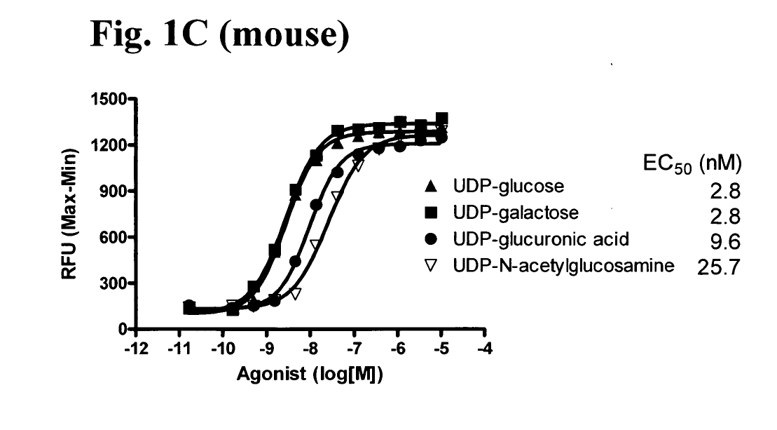

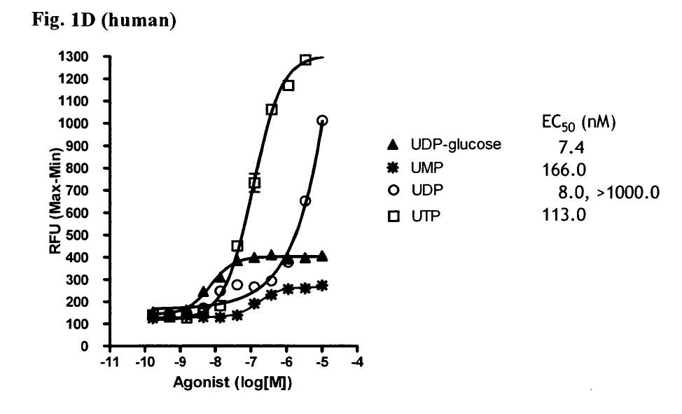

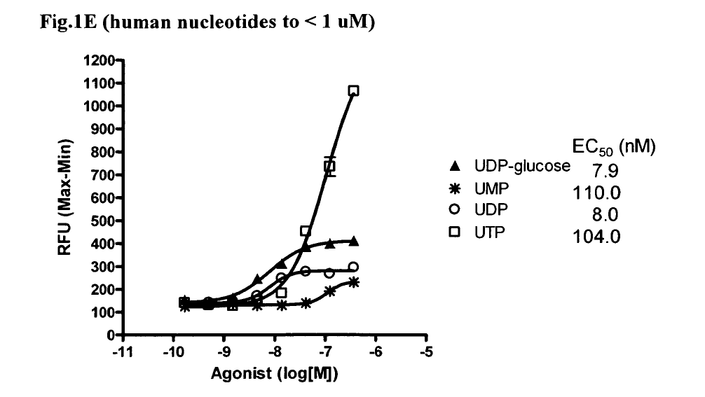

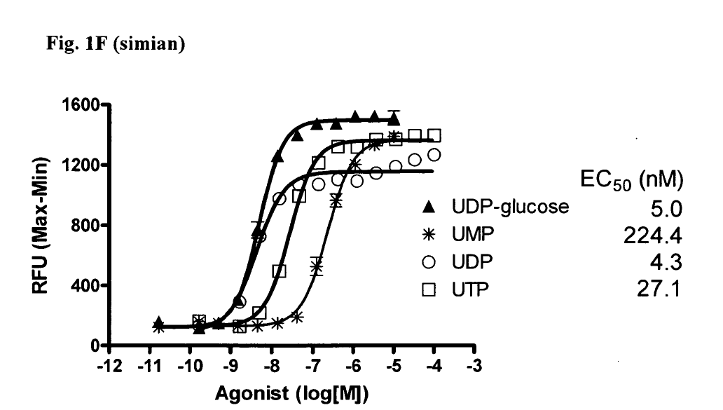

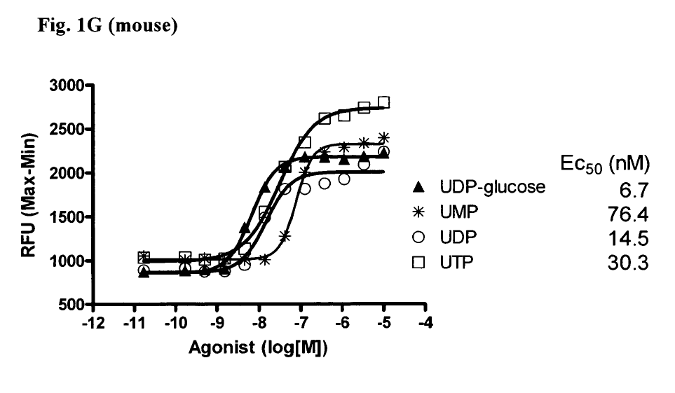

As shown in FIGS. 1A, 1B, and 1C, UDP-glucose, UDP-galactose, UDP-glucuronic acid, and UDP-N-acetylglucosamine all activated human, Chimpanzee, and mouse P2RY14 with similar rank orders of activation. But as shown in FIGS. 1D, 1E, 1F and 1G, UDP-glucose, UMP, UDP, and UTP uncoupled to glucose also activated P2RY14 in the HEK cells expressing human, simian, or mouse P2RY14 in combination with Gqi5. UTP and UDP were able to elicit a much larger Ca²⁺ response as compared to UDP-glucose and UMP in HEK cells expressing human P2RY14 (FIG. 1D), but not in HEK cells expressing simian and mouse P2RY14 (FIGS. 1F and 1G, respectively). Furthermore, in HEK cells expressing human P2RY14, the response to UDP was unique in that the agonist dose curve was biphasic in nature, with a small but reproducible Ca²⁺ signal detected at UDP concentrations of less than 1 μM followed by a larger signal detected at concentrations of greater than 1 μM (See FIG. 1D). FIG. 1E shows the results of FIG. 1D but wherein the UDP results are graphed using the data less than 1 μM. This biphasic UDP dose response was also detected, albeit at a noticeably reduced level, in HEK cells expressing the simian and mouse receptors. These results are in contrast to those published by Chambers et. al. where they were unable to demonstrate agonist activity for UMP, UDP, or UTP on P2RY14 expressed in yeast or HEK-293 cells.

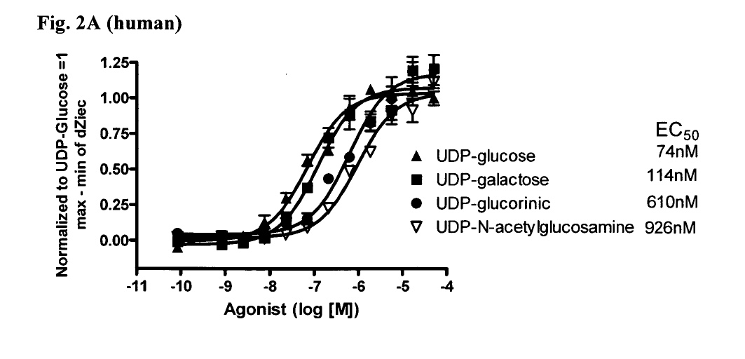

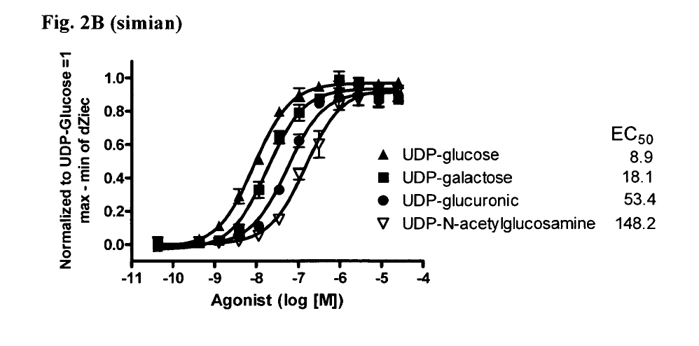

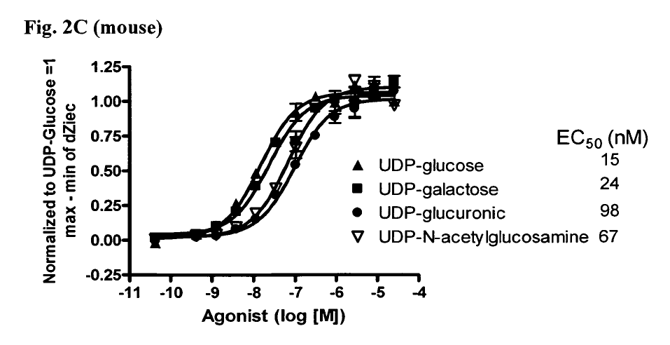

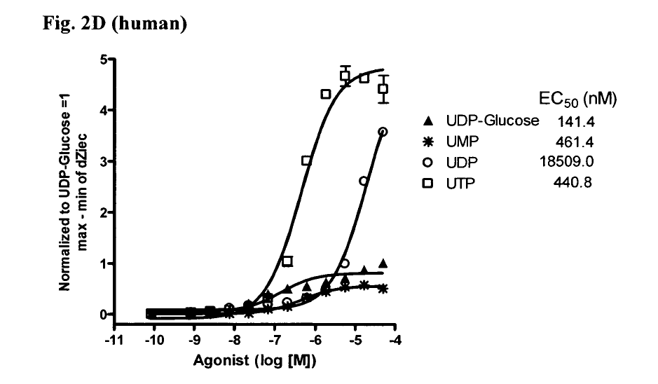

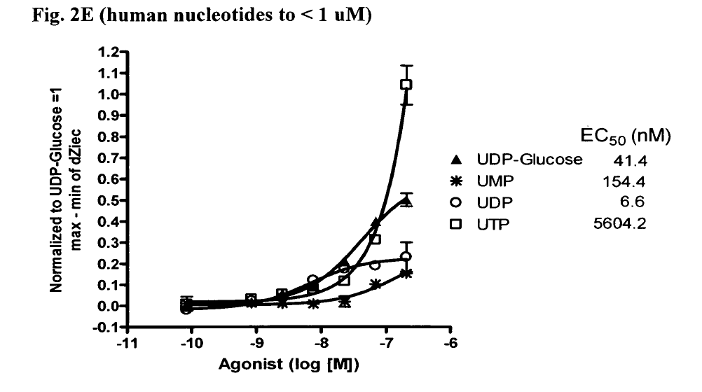

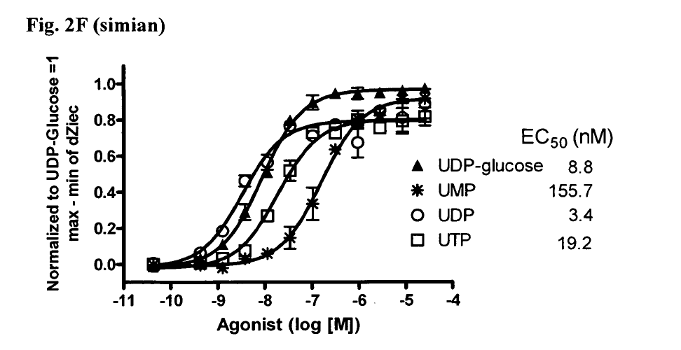

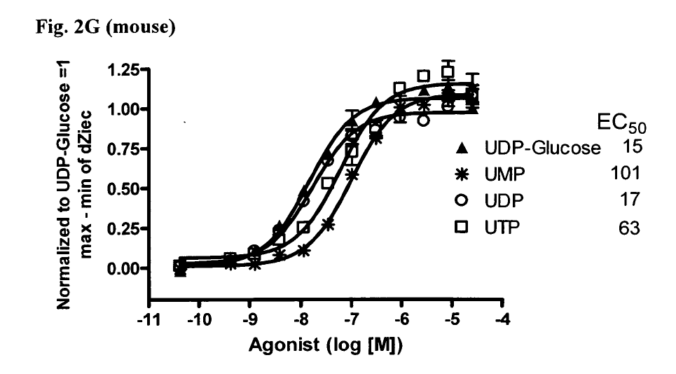

The Cellkey System™ (MDS Sciex), a novel, live cell, label-free, real-time technology, was used to examine P2RY14 activation following agonist stimulation. The CellKey System™ is based on cellular dielectric spectroscopy (CDS) and monitors changes in impedance of the applied electrical current through the cell monolayer upon receptor activation. Using the CellKey System™ and HEK cells expressing human, simian, or mouse P2RY14 in combination with Gqi5, we have been able to confirm not only UDP-glucose, UDP-galactose, UDP-glucuronic, and UDP-N-acetylglucosamine-mediated signaling (FIGS. 2A, 2B, and 2C), but also UMP, UDP, and UTP-mediated signaling (FIGS. 2D, 2E, 2F, and 2G). FIG. 2E shows the results of FIG. 2D but wherein the nucleotide results are graphed using the data less than 1 μM. Therefore, we show for the first time that the sugar moiety is not required for activation of P2RY14 in our HEK cells expressing P2RY14 in combination with Gqi5 using distinct assay technologies. This is in contrast to the results described in Fricks et al. (The FASEB J. 21: 568.14 (2007), which suggested that UDP was a competitive antagonist for the human P2YR14 receptor but a full agonist for the rat P2YR14 receptor.

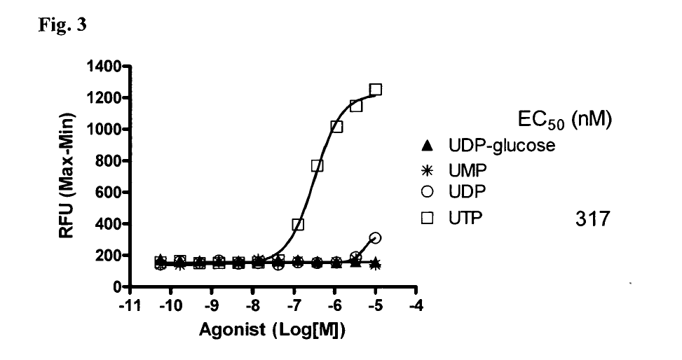

UDP-glucose, UMP, and UDP selectively activate P2RY14 (stimulate Ca²⁺ release in HEK cells expressing P2RY14, FIGS. 1D, 1E, 1F, 1G, 2D, 2E, 2F, and 2G) but do not stimulate Ca²⁺ release in cells not expressing P2RY14 (FIG. 3). In contrast, UTP is able to stimulate Ca²⁺ release in a dose-dependent manner in HEK cells not expressing P2RY14 and therefore is not entirely selective for P2RY14 (FIG. 3).

Since UTP is able to activate P2RY2 and P2RY4 and mediate Ca²⁺ signaling through coupling to Gq (Sak and Webb, Arch. Biochem. Biophys. 397: 131-136 (2002)), and both receptors have been shown to be expressed at both the RNA and protein levels in HEK cells (Fischer et. al. Naunyn-Schmiedeberg's Arch Pharmacol 371: 466-472 (2005)), the Ca²⁺ response to UTP observed in HEK cells expressing P2RY14 and Gqi5 presumably results from activation of P2RY2 and/or P2RY4 and/or another unidentified receptor and P2RY14.

Based on these observations, in addition to UDP-glucose, UMP and UDP may be used as agonists for P2RY14 in cell based assays utilized for screening to identify selective P2RY14 modulators or antagonists.

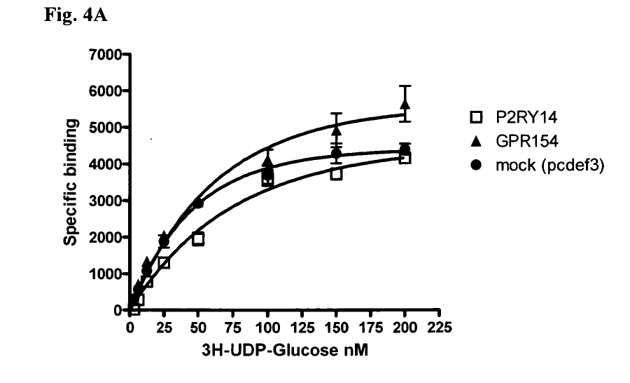

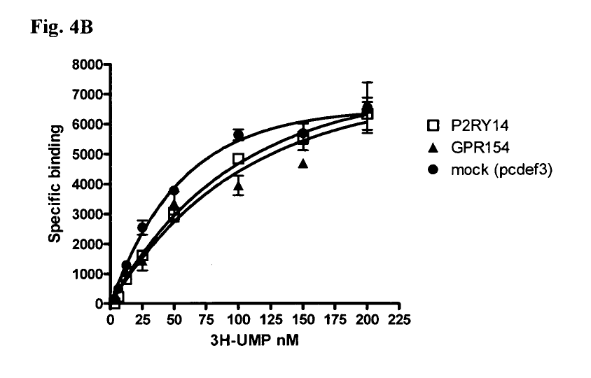

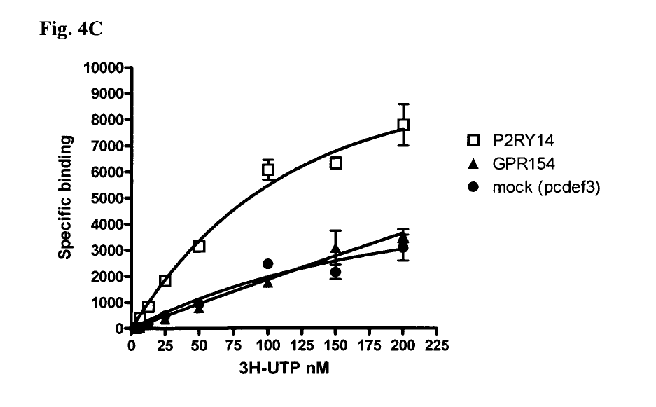

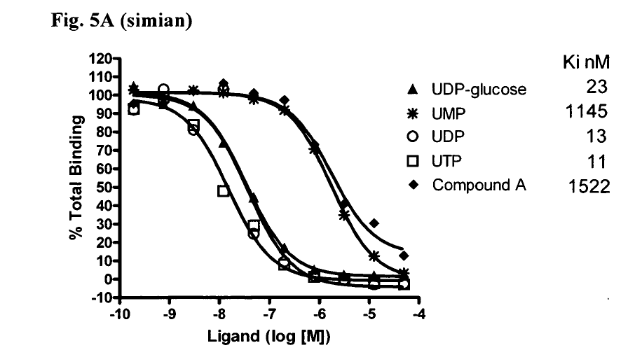

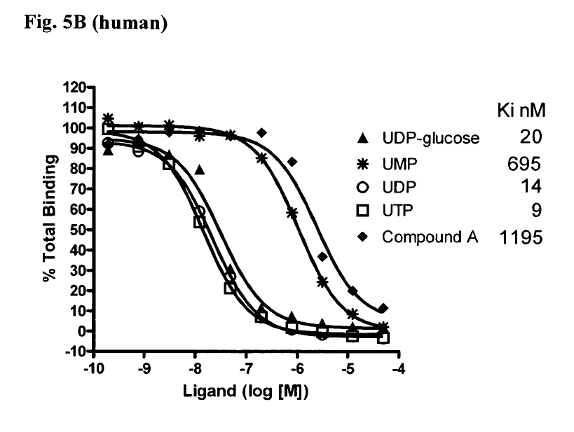

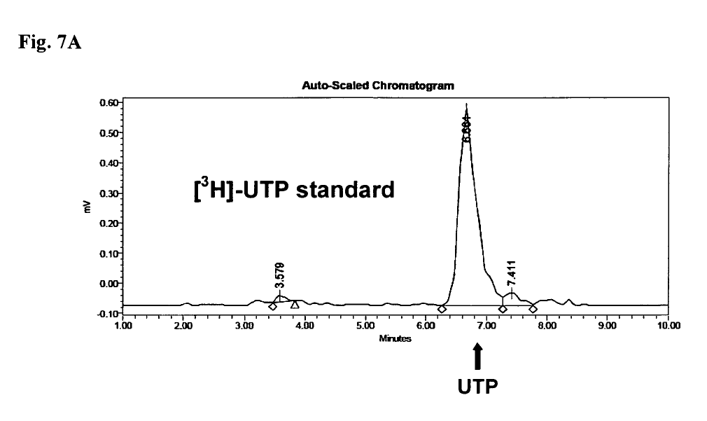

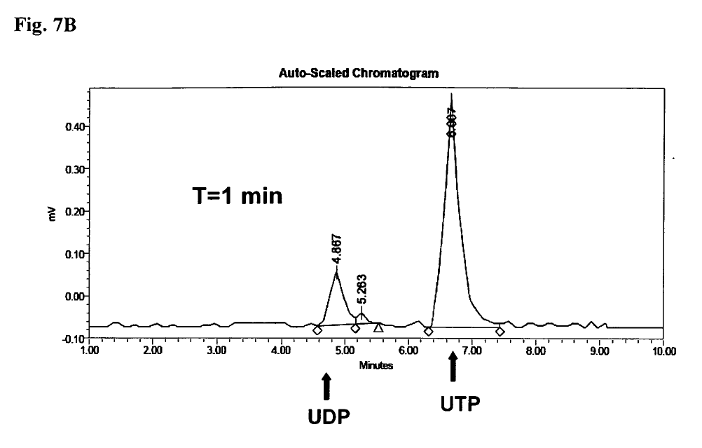

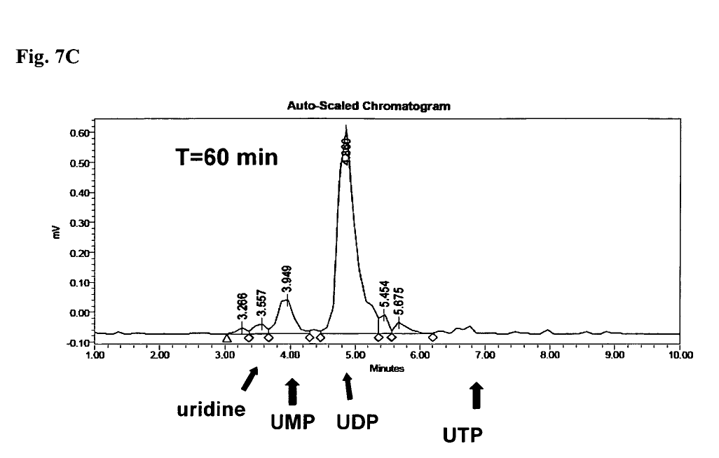

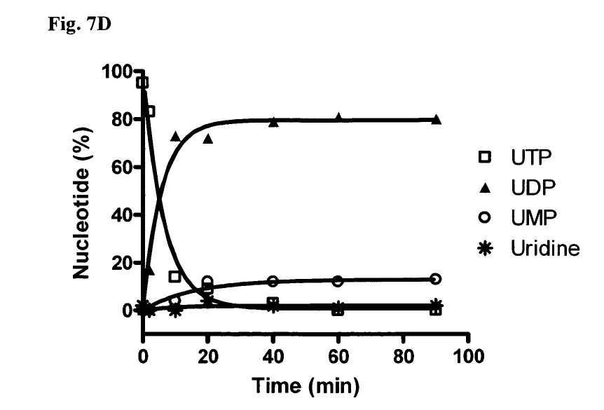

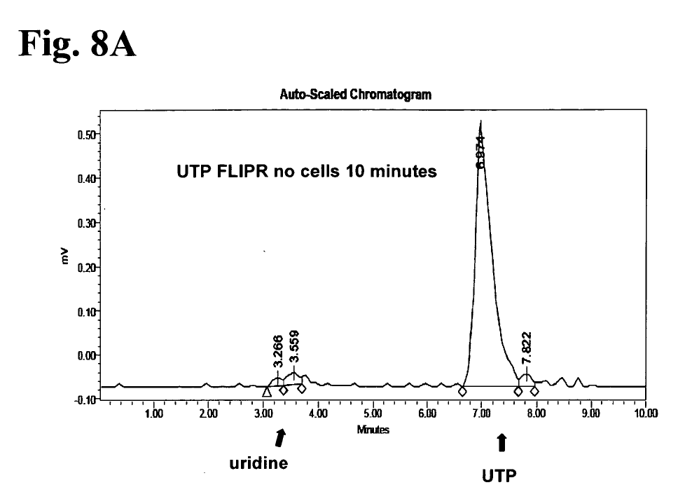

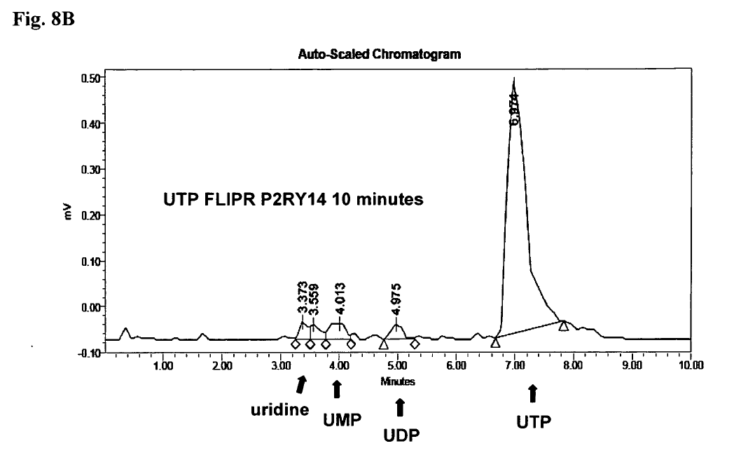

An attempt to establish a membrane filtration binding assay using [³H]-UDP-glucose as radioligand was unsuccessful. Based on the observation that the sugar moiety was not essential to activate P2RY14 in the functional assay, a membrane filtration binding assay using commercially available [³H]-UMP or [³H]-UTP as radioligand was attempted. Although no specific binding window was observed in HEK cells expressing simian P2RY14 using [³H]-UDP-glucose (FIG. 4A) or [³H]-UMP (FIG. 4B), a specific binding window using commercially available [³H]-UTP as radioligand for both simian (FIG. 4C) and human (data not shown) was evident. In time course membrane filtration experiments, [³H]-UTP was shown to be converted predominantly to [³H]-UDP (FIG. 7A-7C) and this conversion occurred within approximately the first 10 minutes of the 60 minute incubation (FIG. 7D). (In contrast, UTP was shown to be stable during the 10 minute cell-based FLIPR assay, confirming that UTP itself is able to activate P2RY14 in the HEK cell line expressing P2RY14 and Gqi5 (FIGS. 8A and 8B)). FIG. 8A is a control with no cells and FIG. 8B uses the HEK cell line expressing P2RY14 and Gqi5. In addition, UDP-glucose, UMP, UDP, and UTP/UDP were able to successfully compete for [³H]-UTP/[³H]-UDP binding in a competition binding assay with similar Ki values for UDP-glucose, UTP/UDP and UDP. UMP was significantly less potent in competing for P2RY14 binding with a Ki of approximately 1000 nM (FIG. 5A). Similar results were seen using membranes expressing human P2RY14 (FIG. 5B). This result indicates that a competitive binding assay that comprises UTP/UDP can be used to identify agents that compete with binding of UTP/UDP for P2RY14.

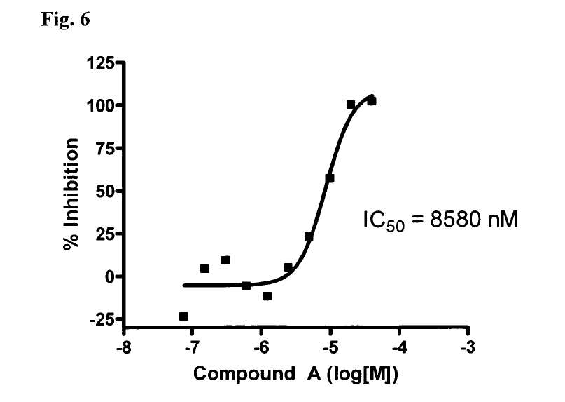

Compound A is a novel P2RY14 antagonist that was identified in the FLIPR cell based assay as being able to inhibit UDP-glucose mediated Ca²⁺ signaling with an IC₅₀ of 8580 nM (FIG. 6). Compound A was also able to successfully compete for [³H]-UTP/[³H]-UDP binding to simian and human P2RY14 with Ki values comparable to UMP (FIGS. 5A and 5B). The UMP, UDP, and UTP scaffold can be utilized by chemists to synthesize potentially novel new antagonists for P2RY14. This could provide novel therapeutics for the treatment of metabolic syndrome.

Therefore, provided is a method for identifying modulators of P2RY14 activity that uses inosine diphosphate, UMP, UDP, or UTP as a P2RY14 agonist. In practice of the invention, a living cell, more typically a population of living cells, such as a liquid culture of living cells that expresses the P2RY14 on the surface thereof is/are contacted with an agent in the presence of inosine diphosphate, UMP, UDP, or UTP. It is then determined whether the agent modulates the biological activity of a P2RY14 protein in the living cell. By way of example, modulation of the biological activity of P2RY14 protein in a living cell can be identified using the method disclosed by Kunapuli et al., Analyt. Biochem. 314: 16-29 (2003). In brief, a vector that includes a nucleic acid molecule encoding a P2RY14 protein is stably introduced into cells, such as HEK cells or CHO cells, and the encoded P2RY14 protein is expressed in the cells. The cells further have a reporter system that is responsive to the activity of the P2RY14 protein, that is, a second component capable of providing a detectable signal in response to the binding of a compound to the P2RY14 protein. An example of the suitable reporter system is a system that uses a chimeric Gqi5 protein, which provides the ability to ascertain P2RY14 activity by measuring Ca²⁺ stimulation. Thereafter, the cells are contacted with a test agent (agonist or antagonist) of P2RY14 in the presence of inosine diphosphate, UMP, UDP, or UTP and the effect of the test agent on the reporter system is measured.

By way of example, a cDNA molecule that encodes a human P2RY14 protein (SEQ ID NO:1) is cloned into an expression vector and co-transfected with a vector encoding a chimeric Gqi5 into cells such as HEK-293 cells so that the transfected cells express P2RY14 protein (SEQ ID NO:1) and chimeric Gqi5. The cells are maintained for several days and then plated into a multi-well format and challenged with various concentrations of UMP, UDP, or UTP and various concentrations of the agent being tested for an effect of the biological activity of the P2RY14 protein. Measurement of Ca²⁺ stimulation in these HEK cells is performed using FLIPR (Molecular Devices, CA, USA) as previously described (Freeman et al., Genomics 78:124-128 (2001)).

Further provided is a binding assay for identifying agents that inhibit binding of UTP/UDP to P2RY14 protein. The method involves using cells having the P2RY14 protein on the surface thereof, or to cell membranes containing the P2RY14 protein and determining the inhibition of binding of a labeled or unlabeled UTP/UDP to the P2RY14 protein in the presence of the agent under conditions that normally permit binding of the UTP/UDP to the P2RY14 protein in the absence of the agent. Agents that inhibit binding of UTP/UDP to P2RY14 protein can be either an antagonist or agonist of the P2RY14 protein, or in some cases not have either agonist or antagonist activity. A functional assay as described herein can be used to determine whether the agent that inhibits binding of UTP/UDP to the P2RY14 protein is an antagonist or agonist of P2RY14 activity or has no effect on P2RY14 activity.

While the examples use human, simian (chimpanzee), and mouse P2RY14, other P2RY14 proteins can be substituted for the human P2RY14 (SEQ ID NO: 1), simian P2RY14 (SEQ ID NO:6), or mouse P2RY14 (SEQ ID NO:5). Other P2RY14 proteins include, but are not limited to, those obtainable from rat (SEQ ID NO:3), dog (SEQ ID NO:4), Rhesus monkey (SEQ ID NO:2), or chicken (SEQ ID NO:7).

While the examples use HEK-293 cells, other cells which may be suitable and which are commercially available, include but are not limited to, L cells L-M(TK-) (ATCC CCL-1.3), L cells L-M (ATCC CCL-1.2), Saos-2 cells (ATCC HTB-85), Raji cells (ATCC CCL-86), CV-1 cells (ATCC CCL-70), COS-1 cells (ATCC CRL-1650), COS-7 cells (ATCC CRL-1651), CHO-K1 cells (ATCC CCL-61), 3T3 cells (ATCC CCL-92), NIH/3T3 cells (ATCC CRL-1658), HeLa cells (ATCC CCL-2), C1271 cells (ATCC CRL-1616), BS-C-1 cells (ATCC CCL-26), MRC-5 cells (ATCC CCL-171), ST2 cells (Riken Cell bank, Tokyo, Japan RCB0224), C3H10T1/2 cells (JCRB0602, JCRB9080, JCRB0003, or IFO50415), and CPAE cells (ATCC CCL-209).

Numerous assays (for example, hundreds or thousands of assays) for assessing the effect of an agent on P2RY14 biological activity in the presence of UMP, UDP, or UTP can be automated and conducted simultaneously. For example, the assays can be automated in a high-throughput sequence format as described, for example, by Kornienko et al., J. Biomol. Screen 9(3):186-195 (2004).

In the practice of the claimed methods, the modulating effect of an agent on P2RY14 biological activity is validated in vivo. The validation step shows that an agent that modulates the biological activity of P2RY14, in vitro, also causes a significant improvement in a phenotype, in vivo, associated with type II diabetes and/or obesity (for example, the agent causes one or more of the following changes: lowers LDL cholesterol, raises HDL cholesterol, lowers body weight, decreases the rate of body weight gain in response to a diet high in fat, decreases insulin resistance, increases glucose tolerance and/or decreases fat pad mass (in rats or mice) in response to a high fat diet).

In another aspect, the present invention provides methods for determining the effect of an agent on P2RY14 activity. The methods of this aspect of the invention each include the steps of (a) observing a change in a P2RY14-mediated response in a living cell, in vitro, in response to an agent in the presence of inosine diphosphate, UMP, UDP, or UTP; and (b) confirming that the observed change in the P2RY14-mediated response occurs in vivo.

In a further aspect, the present invention provides methods for identifying an agent that ameliorates a symptom of type II diabetes or obesity. The methods of this aspect of the invention include the steps of: (a) contacting a living cell, in vitro, with an agent, wherein the living cell expresses a P2RY14 protein having a biological activity on the surface of the cell in the presence of inosine diphosphate, UMP, UDP, or UTP; (b) determining whether the agent modulates the biological activity of the P2RY14 protein in the living cell; and (c) determining, in vivo, whether the chemical agent ameliorates a symptom of type II diabetes or obesity. Examples of symptoms of type II diabetes and/or obesity include increased insulin resistance, increased body mass, and a decreased rate of glucose clearance from the blood stream.

The following examples are intended to promote a further understanding of the present invention.

### EXAMPLE 1

Illustrates construction of HEK cells expressing P2RY14 and chimeric Gqi5 and development of a whole cell functional FLIPR assay for P2RY14

Stable HEK clonal cell lines expressing P2RY14 and the chimeric G protein Gqi5 were constructed. The chimeric Gqi5 forces the coupling of P2RY14 through the Gq (calcium) pathway and allows for monitoring of calcium signaling using a calcium binding fluorescent dye and a FLIPR (fluorometric imaging plate reader, MDS Sciex). Vector pCEP4 comprising the chimeric G_{qi5} was obtained from Molecular Devices, Sunnyvale, Calif. (Cat. No. RD-PGQI5).

The human P2RY14 was PCR amplified from the I.M.A.G.E. clone obtained from Invitrogen, Carlsbad, Calif. (Cat. #5265592) using primer hGPR105_{—}5′_KpnI AAAGGTACCGCCACCATGATCAATTCAACCTCCAC (SEQ ID NO: 14) AND PRIMER HGPR105_{—}3′_NotI AAAGCGGCCGCTCACAAAGTATCTGTGCTTTCAAG (SEQ ID NO:15) to produce the nucleotide fragment shown in SEQ ID NO:8. The codon encoding glutamate at position 54 in the wild-type human P2RY14 was changed to code for lysine. The PCR conditions were 50 ng of the I.M.A.G.E. clone #5265592, 1× final concentration of cloned Pfu DNA polymerase reaction buffer, 300 μM each of dATP, dCTP, dTTP, and dGTP, 300 nM of each primer, dH₂O to a final volume of 50 μL, and 2.5 units PfuTurbo® hotstart DNA polymerase (Stratagene, La Jolla, Calif.). The PCR reaction was done in two parts. In the first part, the reaction was 5 cycles at 94° C. for 30 seconds, 65° C. for 30 seconds, and 72° C. for 90 seconds. In the second part, the reaction was 25 cycles at 94° C. for 30 seconds, 70° C. for 30 seconds, 72° C. for 90 seconds. The last cycle was followed by 7 minutes at 72° C. The PCR amplified human P2RY14 was digested with KpnI and NotI and cloned into a pcDNA3.1 (+) vector previously digested with KpnI and NotI to place the DNA fragment in the proper orientation downstream of the CMV promoter. The nucleic acid encoding the human P2RY14 was under the control of the CMV promoter.

The simian (chimpanzee) P2RY14 nucleic acid fragment was synthesized based upon the Ensembl Gene ID ENSPTRG00000030093 by Bio S & T, Montreal, Quebec, Canada, to be flanked with KpnI and NotI restriction enzyme sites. The nucleotide sequence of the simian P2RY14 is shown in SEQ ID NO: 10. The DNA fragment was digested with KpnI and NotI and then cloned into a pcDEF3 vector previously digested with KpnI and NotI to place the DNA fragment in the proper orientation downstream of the human EF-1a promoter. The nucleic acid encoding the simian P2RY14 was under the control of the human EF-1a promoter.

The mouse P2RY14 was subcloned unidirectionally from the I.M.A.G.E. clone from Invitrogen (Cat. No. 6314145; in vector pCMV-SPORT6.1) by digesting the clone with HindIII and NotI and then cloning the DNA fragment containing the P2RY14 into a pcDNA3.1(+) vector digested with HindIII and NotI to place the DNA fragment in the proper orientation downstream of the CMV promoter. The nucleotide sequence of the mouse P2RY14 is shown in SEQ ID NO: 12. The nucleic acid encoding the mouse P2RY14 was under the control of the CMV promoter.

For construction of the HEK cell line expressing P2RY14 and Gqi5, 2 μg of plasmid comprising a nucleic acid encoding human, simian, or mouse P2RY14 and 0.2 μg of plasmid pCEP4 comprising the nucleic acid encoding the chimeric Gqi5 were transfected into 0.4×10⁶ HEK ebna cells using FUGENE 6 (Roche), according to the manufacturer's instructions. Cells were grown in DMEM medium containing 25 mM HEPES, pH 7.4 and 10% FBS, 50 units/ml penicillin/50 μg/ml streptomycin, 2 mM glutamine and 1 mM sodium pyruvate at 37° C. in humidified air containing 5% CO₂. Two days post-transfection, cells were grown under selection in the DMEM medium as indicated above and supplemented with 200 μg/ml Hygromycin B and 250 μg/ml geneticin.

P2RY14 expressing clonal cells were selected for by UDP-glucose-induced Ca²⁺ release using FLIPR, as follows. Briefly, 12,500 HEK/P2RY14/Gqi5 expressing cells were plated in 25 μl onto 384 well, poly D lysine coated plates in DMEM containing 10% FBS. Cells were incubated overnight at 37° C. and 5% CO₂ to form a monolayer. On the following day, 30 μl of fluorescent no-wash dye was added to the cell monolayer and the plate was incubated for 60 minutes at 37° C., 5% CO₂.

For agonist assays, 6.12 μl of 10× agonist solution was added to the cell/dye incubation and Ca²⁺ signaling was monitored by FLIPR. For antagonist assays, 250 nl of compound in 100% DMSO was added to cell/dye mixture; following a 20 minute incubation at room temperature, 6.12 μl of 10× agonist solution at EC80 in HBSS containing 20 mM Hepes was added to cells and Ca²⁺ signaling was monitored by FLIPR. Quantitation of the % stimulation or inhibition of Ca²⁺ signaling by agonist or antagonist, respectively, was calculated using the Max-Min fluorescent signal detected.

FIGS. 1A, 1B, and 1C show that UDP-glucose (and other UDP-sugars) are agonists of human, simian, and mouse P2RY14. FIGS. 1D, 1F, and 1G show that in addition to UDP-glucose, UMP, UDP, and UTP also are agonists of human, simian, and mouse P2RY14. It is interesting to note that UTP and UDP were able to elicit a much larger Ca²⁺ response as compared to UDP-glucose and UMP in HEK cells expressing human P2RY14 (FIG. 1D), but not in HEK cells expressing simian or mouse P2RY14 (FIGS. 1F and 1G, respectively). FIG. 1E shows the results of FIG. 1D but wherein the UDP results are graphed using the data less than 1 μM. FIG. 1E shows that at the nanomolar level UDP in the human has agonist activity.

### EXAMPLE 2

This example uses the CellKey™ System Live Cell Functional Assay to show that UMP, UDP, and UTP are P2RY14 agonists.

HEK293-Gqi5-P2RY14 cells were seeded into a CellKey™ standard 96-well microplate at 1.25×10⁵ cells/100 μl per well and incubated for 18 hours at 37° C. and 5% CO₂, in Dulbecco's modified Eagle medium containing 10% FBS, 10-mM HEPES and 50 units/ml penicillin/50 μg/ml streptomycin. On the following day, growth medium was removed from the cell monolayer and replaced with 135 μl of HBSS pH 7.4 containing 20 mM Hepes for a 30 minutes pre-incubation period at room temperature. Baseline measurements were made for 5 minutes at 28° C. prior to ligand addition. For agonist assays, 15 μl of 10× agonist diluted in HBSS pH 7.4 containing 20 mM HEPES was added to the cell monolayer simultaneously using the 96 multichannel head from the CellKey™ System. Cellular response was monitored for 10 minutes to assess cellular response after ligand addition.

The results are shown in FIGS. 2A-2G and confirm not only that UDP-glucose, UDP-galactose, UDP-glucuronic and UDP-N-acetylglucosamine mediate P2RY14 signaling (See FIGS. 2A, 2B, and 2C), but also UMP, UDP, and UTP mediate P2RY14 signaling (See FIGS. 2D, 2E, 2F, and 2G). FIG. 2E shows the results of FIG. 2D but wherein the nucleotide results are graphed using the data less than 1 μM. In a FLIPR assay, UDP-glucose, UMP, and UDP, do not stimulate Ca²⁺ release in cells not expressing P2RY14 (FIG. 3). Thus, UDP-glucose, UMP, and UDP appear to selectively activate P2RY14. However, FIG. 3 also shows that in contrast to UDP-glucose, UMP, and UDP, UTP is able to stimulate Ca²⁺ release in a dose-dependent manner in HEK cells not expressing P2RY14. Therefore, the results show that while UDP-glucose, UMP, and UDP do appear to be selective for P2RY14, UTP appears not to be entirely selective for P2RY14.

### EXAMPLE 3

In this example, simian and human P2RY14 binding assays were performed with UMP, UDP, and UTP.

HEK 293 EBNA cell pellets, transiently transfected with Chimpanzee or human P2RY14 cDNA in pdy7 vector, or pdy7 vector only, were obtained from Biotechnology Research Institute (BRI) from the National Research Council of Canada (NRC) (Transient Gene Expression in HEK293 Cells: Peptone Addition Posttransfection Improves Recombinant Protein Synthesis Pham et al., Animal Cell Technology Group, Bioprocess Sector, Biotechnology Research Institute, National Research Council Canada. 6100 Royalmount Ave., Montreal (Quebec) Canada H4P 2R2; Biotechnol. Bioengineer. 90(3): May 5, 2005).

Membrane preparations were made by N2 cavitation using a Parr unit. Cells were thawed on ice and resuspended in 10 mM Hepes/1 mM EDTA, pH 7.4 (KOH) containing 1× protease inhibitor Complete (Roche # 1697498) at a maximum concentration of 10⁸ cells per ml. Cell were Dounce homogenized (size B, 10 strokes) and then added to the cavitation unit. Pressure was adjusted to 800 psi and cells were left under pressure for 30 minutes. Broken cells were released drop by drop and spun at 1000 g for 10 minutes. Supernatent was spun at 160,000 g (rotor type 60 Ti, 40000 rpm for 30 min). Pellet was resuspended in 10 mM HEPES/KOH pH 7.4, 1 mM EDTA in ⅕ the original volume, dounce homogenized (size A, 10 strokes), aliquoted, and frozen at −80° C.

Binding assays were performed in 10 mM Hepes pH 7.4 (KOH), 5 mM MnCl₂, 5 mM MgCl₂ buffer. Membranes (10 μg/well for human P2RY14 and 30 μg/well for simian P2RY14) were incubated with 10 nM [5.6-³H] Uridine 5′-triphosphate (UTP), ammonium salt from Amersham Biosciences (# TRK412) in presence of compounds diluted in DMSO (2% final in assay), in a total volume of 100 μl. 50 minutes (human P2RY14) or 60 minutes (simian) P2RY14) incubations were performed at room temperature with shaking. Binding in wells containing 2 μl DMSO only (no drug) represents total binding while binding measured in presence of 1000× cold UTP/UDP in DMSO corresponds to non-specific binding. Incubation mixtures were filtered on GF/C Packard filter membranes (#6065174) and washed with cold 10 mM Hepes/KOH buffer at pH 7.4 (2×3.7 ml) using a Tomtek Mach III harvester. GF/C filters were subsequently dried at 55° C. in a vacuum oven for 1 hour before addition of 25 μl/well Ultima Gold F scintillation fluid from Packard (#6013179) and counted in a Wallac Microbeta counter normalized for ³H (2 min/well). Counts (ccpms) generated by non specific binding were subtracted from total counts, to give specific binding which was expressed as % of total binding and K is were calculated by data analyzer using the inflection point of the dose dependant sigmoidal curve generated for each compound. K_{d}s for [³H]-UTP/[³H]-UDP in the human and simian assays were determined by Scatchard analysis and are as follow: 10.03 nM for human P2RY14 and 10.25 nM for simian P2RY14. Binding of [³H]-UTP/[³H]-UDP to HEK 293 EBNA cell membranes from cells mock transfected with the pdy7 vector only were used to demonstrate [³H]-UTP/[³H]-UDP binding specificity to P2RY14. To examine the stability of [³H]-UTP in the membrane filtration binding assay, 100 ul aliquots of the reaction mixture were extracted at 2, 10, 20, 40, 60 and 90 minutes with 100 uL of cold 80 mM EDTA solution (pH 8) and 200 uL of cold Acetonitrile. After mixing, proteins were precipitated by centrifugation at 13000 rpm for 10 minutes at 4° C. 50 ul of the supernatant was injected onto a Waters 2695 HPLC system with a Zorbax ion exchange SAX column (5 um, 4.6×256 mm) and a Flow Scintillation Analyzer for radioactive detection using a mobile phase consisting of 10:90 50 mM KH₂PO4/750 mM KH₂PO₄, pH3.0. After 0.5 min at 10:90 50 mM KH₂PO₄/750 mM KH₂PO₄, a gradient was applied in order to increase to 100% 750 mM KH₂PO₄ at 5 min, after which this composition was maintained for an additional 3 min. Samples were kept at 4° C. prior to HPLC analysis

FIGS. 4A and 4B show that no specific binding window was observed in HEK cells expressing simian P2RY14 using [³H]-UDP-glucose or [³H]-UMP, respectively; however, FIG. 4C shows that a specific binding window using [³H]-UTP/[³H]-UDP as radioligand was evident.

FIG. 5A shows that UDP-glucose, UMP, UDP, and UTP/UDP were able to successfully compete for [³H]-UTP/[³H]-UDP binding in a competition binding assay with similar Ki values for UDP-glucose, UTP/UDP and UDP. As shown, UMP was significantly less potent in competing for P2RY14 binding with a Ki of approximately 1000 nM. Similar results were seen using membranes expressing human P2RY14 (FIG. 5B). FIG. 7A-C shows that [³H]-UTP is converted predominantly to [³H]-UDP during the 60 minute assay incubation. FIG. 7D shows that [³H]-UTP is converted to [³H]-UDP during the first 10 minutes of the reaction and remains stable for up to 90 minutes.

### EXAMPLE 4

This example shows that compound A, which was identified in a FLIPR cell-based assay for identifying antagonists performed essentially as described in Example 1, is a novel P2RY14 antagonist. FIG. 6 shows that compound A was able to inhibit UDP-glucose mediated Ca²⁺ signaling with an IC₅₀ of 8580 nM. Compound A was also able to successfully compete for [³H]-UTP/[³H]-UDP binding to simian and human P2RY14 with Ki values comparable to UMP (See FIGS. 5A and 5B).

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1: A method for identifying agents that modulate pyrimidinergic receptor P2Y, G-protein coupled, 14 (P2RY14) protein activity comprising:
(a) contacting a cell expressing on the surface thereof the P2RY14 protein, the P2RY14 protein being associated with a second component capable of providing a detectable signal in response to the activation of P2RY14 protein, with an agent to be screened in the presence of a labeled or unlabeled nucleotide selected from the group consisting of inosine diphosphate, UMP, UDP, and UTP; and
(b) determining whether the agent modulates the P2RY14 protein activity by measuring the level of a signal generated from the interaction of the agent with the P2RY14 protein, wherein a change in the signal from that obtained in the absence of the agent identifies the agent as a modulator of the P2RY14 protein activity.

2-3. (canceled)

4: The method of claim 1, wherein the P2RY14 protein has an amino acid sequence selected from the group consisting of SEQ ID NO: 1, SEQ ID NO:2, SEQ ID NO:3, SEQ ID NO:4; SEQ ID NO:5, SEQ ID NO:6, and SEQ ID NO:7.

5: The method of claim 1, wherein the P2RY14 protein has the amino acid sequence set forth in SEQ ID NO: 1.

6: The method of claim 1, wherein the P2RY14 protein has the amino acid sequence set forth in SEQ ID NO:2.

7: The method of claim 1 wherein the second component comprises a chimeric Gqi5 protein.

8: The method of claim 1 wherein the detectable signal is calcium signaling.

9: The method of claim 1 wherein when the signal is decreased, the agent is an antagonist of the P2RY14 protein, and when the signal is increased, the agent is an agonist of the P2RY14 protein.

10-15. (canceled)

16: A method for identifying agents that competitively inhibit binding of UTP/UDP to a pyrimidinergic receptor P2Y, G-protein coupled, 14 (P2RY14) protein, comprising:
(a) measuring binding of a labeled or unlabeled UTP/UDP to cells having the P2RY14 protein on the surface thereof, or to cell membranes containing the P2RY14 protein, in the presence of the agent under conditions to permit binding of the UTP/UDP to the P2RY14 protein in the absence of the agent; and
(b) determining the amount of the UTP/UDP bound to the P2RY14 protein, wherein an agent that causes a reduction of binding of the UTP/UDP to the P2RY14 protein competitively inhibits the binding of the UTP/UDP to the P2RY14 protein.

17-18. (canceled)

19: The method of claim 16, wherein the P2RY14 polypeptide has an amino acid sequence selected from the group consisting of SEQ ID NO: 1, SEQ ID NO:2, SEQ ID NO:3, SEQ ID NO:4; SEQ ID NO:5, SEQ ID NO:6, and SEQ ID NO:7.

20: The method of claim 16, wherein the P2RY14 polypeptide has the amino acid sequence set forth in SEQ ID NO: 1.

21: The method of claim 16, wherein the P2RY14 polypeptide has the amino acid sequence set forth in SEQ ID NO:2.

22. A method for identifying agents that bind a pyrimidinergic receptor P2Y, G-protein coupled, 14 (P2RY14) protein and modulate P2RY14 activity comprising:
(a) contacting a cell expressing on the surface thereof the P2RY14 protein, the P2RY14 protein being associated with a second component capable of providing a detectable signal in response to the activation of P2RY14 protein, with an agent to be screened in the presence of a labeled or unlabeled nucleotide selected from the group consisting of inosine diphosphate, UMP, UDP, and UTP;
(b) determining whether the agent modulates the P2RY14 protein activity by measuring the level of a signal generated from the interaction of the agent with the P2RY14 protein, wherein a change in the signal from that obtained in the absence of the agent identifies the agent as a modulator of the P2RY14 protein activity;
(c) contacting cells having the P2RY14 protein on the surface thereof, or cell membranes containing the P2RY14 protein, with the agent from step (b) in the presence of labeled or unlabeled UTP/UDP under conditions to permit binding of the UTP/UDP to the P2RY14 protein in the absence of the agent;
(d) measuring binding of a labeled or unlabeled UTP/UDP to the cells or membranes; and
(e) determining the amount of the UTP/UDP bound to the P2RY14 protein, wherein an agent that causes a reduction of binding of the UTP/UDP to the P2RY14 protein binds the P2RY14 protein and modulates P2RY14 protein activity.

23: The method of claim 22 wherein when the signal is decreased, the agent is an antagonist of the P2RY14 protein, and when the signal is increased, the agent is an agonist of the P2RY14 protein.

24-25. (canceled)


SEQUENCE LISTING (13 sequences)

SEQ ID NO: 1 - protein, 338 aa, Homo sapiens
MINSTSTQPPDESCSQNLLITQQIIPVLYCMVFIAGILLNGVSGWIFFYVPSSESFIIYLKNIVIADFVMSLTFPFKILGDSGLGPWQLNVFVCRVSAVLFYVNMYVSIVFFGLISFDRYYKIVKPLWTSFIQSVSYSKLLSVIVWMLMLLLAVPNIILTNQSVREVTQIKCIELKSELGRKWHKASNYIFVAIFWIVFLLLIVFYTAITKKIFKSHLKSSRNSTSVKKKSSRNIFSIVFVFFVCFVPYHIARIPYTKSQTEAHYSCQSKEILRYMKEFTLLLSAANVCLDPIIYFFLCQPFREILCKKLHIPLKAQNDLDISRIKRGNTTLESTDTL

SEQ ID NO: 2 - protein, 339 aa, Macaca mulatta
MMINSTSTQPPDESCSKNLLITQQIIPVLYCAVFIAGILLNGVSGWIFFYVPSSKSFIIYLKNIVIADFVMSLTFPFKILSDSGLGPWQLNVFVCRVSAVLFYVNMYVSIVFFGLISFDRYYKIVKPLWTSFIQSVSYSKLLSVIVWMLMLLLAVPNIILTNQSVREVTNIKCIELKSELGRKWHTASNYIFVGIFWIVFLLLIVFYTAITKKIFKSHLKSSRNSTSVKKKSSRNIFSIVFVFFVCFVPYHIARIPYTKSQTEAHYSCQSKEILRYMKEFTLLLSAANVCLDPIIYFFLCQPFREILCKKLHIPLKAQNDLDISRTKRGNTTLESIDTL

SEQ ID NO: 3 - protein, 305 aa, Rattus norvegicus
MDNTTTTEPPKQPCTRNTLITQQIIPMLYCVVFITGVLLNGISGWIFFYVPSSKSFIIYLKNIVVADFLMGLTFPFKVLSDSGLGPWQLNVFVFRVSAVIFYVNMYVSIAFFGLISFDRYYKIVKPLLVSIVQSVNYSKVLSVLVWVLMLLLAVPNIILTNQSVKDVTNIQCMELKNELGRKWHKASNYVFVSIFWIVFLLLTVFYMAITRKIFKSHLKSRKNSISVKRKSSRNIFSIVLAFVACFAPYHVARIPYTKSQTEGHYSCQAKETLLYTKEFTLLLSAANVCLDPISISSYASRLEKS

SEQ ID NO: 4 - protein, 340 aa, Canis familiaris
MTNSSTTQPPEESCSRNTVITQQIVPVLYLVVFVAGILLNGVSGWIFFYVPSSKSFIVYLKNIVVADFLMSLTFPFKILADSGLGPWRIHVFVCRVSAVLFYVNMYVSIVFFGFISFDRYYKIVKPLLTSFIHSVNYSKLLSVMVWVLMLLLAVPNIILTNQNVSNVKVTRIKCVTLKNELGLKWHKASNYIFVGIFWIVFFLLIFFYTAITRKIFKSHLKSRRNSISVKRKSSRNIFSIMFVFFVCFVPYHTARIPYTQSQTEAHYSCRSKEILLYVKEFTLLLSAANVCLDPIIYFFLCQPFRETLCKKLHIPFKAQHDSETSKTKRENTTHESTDTL

SEQ ID NO: 5 - protein, 338 aa, Mus musculus
MNNSTTTDPPNQPCSWNTLITKQIIPVLYGMVFITGLLLNGISGWIFFYVPSSKSFIIYLKNIVVADFLMGLTFPFKVLGDSGLGPWQVNVFVCRVSAVIFYVNMYVSIVFFGLISFDRYYKIVKPLLTSIVQSVNYSKLLSVLVWMLMLLLAVPNIILTNQGVKEVTKIQCMELKNELGRKWHKASNYIFVSIFWVVFLLLIVFYTAITRKIFKSHLKSRKNSTSVKRKSSRNIFSIVLVFVVCFVPYHIARIPYTKSQTEGHYSCRTKETLLYAKEFTLLLSAANVCLDPIIYFFLCQPFREVLNKKLHMSLKVQNDLEVSKTKRENAIHESTDTL

SEQ ID NO: 6 - protein, 338 aa, Pan troglodytes
MINSTSTQPPDESCSQNLLITQQIIPVLYCVVFIAGILLNGVSGWIFFYVPSSKSFIVYLKNIVIADFVMSLTFPFKILGDSGLGPWQLNVFVCRVSAVLFYVNMYVSIVFFGLISFDRYYKIVKPLWTSFIQSVSYSKLLSVIVWMLMLLLAVPNIILTNQSVREVTQIKCIELKSELGRKWHKASNYIFVAIFWIVFLLLIVFYTAITKKIFKSHLKSSRNSTSVKKKSSRNIFSIVFVFFICFVPYHIARIPYTKSQTEAHYSCQSKEILRYMKEFTLLLSAANVCLDPIIYFFLCQPFREILCKKLHIPLKAQNDLDISRIKRGNTTLESTDTL

SEQ ID NO: 7 - protein, 337 aa, Gallus gallus
MFNSSTNSSGSSCSNSTVITRAVIPLIYCLIFVVGLLLNGVAAWIFLCISSKKSFIVYLKNIVVADLLMSLTFPFKILADSEIAPPQLNTFVCRYSAVVFYANMYIGISFFGLIGFDRYYKIVKPLFTSFVHTVHYSKVISAIIWLLITFITLPNMILTNEISKANDSRTCIGLKSELGKQWHEVSSYICTGIFWIVFLLLIIFYTSISKKIYSSYKKFRRNSDSAKRKTSRNIFTIMFVFVLCFVPYHFCRIPYTLSQTSPQFNCRSQRALFLAKEFTLLLSAANVCLDPIIYFFLCQPFRERLYQKLHLQLKTSGEAEITKSRRSNTLQESVNIL

SEQ ID NO: 8 - DNA, 1037 bp, Artificial Sequence
  Artificial sequence encodes human P2RY14 receptor
ggtaccgccaccatgatcaattcaacctccacacagcctccagatgaatcctgctctcagaacctcctgatcactcagcagatcattcctgtgctgtactgtatggtcttcattgcaggaatcctactcaatggagtgtcaggatggatattcttttacgtgcccagctctaagagtttcatcatctatctcaagaacattgttattgctgactttgtgatgagcctgacttttcctttcaagatccttggtgactcaggccttggtccctggcagctgaacgtgtttgtgtgcagggtctctgccgtgctcttctacgtcaacatgtacgtcagcattgtgttctttgggctcatcagctttgacagatattataaaattgtaaagcctctttggacttctttcatccagtcagtgagttacagcaaacttctgtcagtgatagtatggatgctcatgctcctccttgctgttccaaatattattctcaccaaccagagtgttagggaggttacacaaataaaatgtatagaactgaaaagtgaactgggacggaagtggcacaaagcatcaaactacatcttcgtggccatcttctggattgtgtttcttttgttaatcgttttctatactgctatcacaaagaaaatctttaagtcccaccttaagtcaagtcggaattccacttcggtcaaaaagaaatctagccgcaacatattcagcatcgtgtttgtgttttttgtctgttttgtaccttaccatattgccagaatcccctacacaaagagtcagaccgaagctcattacagctgccagtcaaaagaaatcttgcggtatatgaaagaattcactctgctactatctgctgcaaatgtatgcttggaccctattatttatttctttctatgccagccgtttagggaaatcttatgtaagaaattgcacattccattaaaagctcagaatgacctagacatttccagaatcaaaagaggaaatacaacacttgaaagcacagatactttgtgagcggccgc

SEQ ID NO: 9 - protein, 338 aa, Artificial Sequence
  Modified human P2RY14 receptor
MINSTSTQPPDESCSQNLLITQQIIPVLYCMVFIAGILLNGVSGWIFFYVPSSKSFIIYLKNIVIADFVMSLTFPFKILGDSGLGPWQLNVFVCRVSAVLFYVNMYVSIVFFGLISFDRYYKIVKPLWTSFIQSVSYSKLLSVIVWMLMLLLAVPNIILTNQSVREVTQIKCIELKSELGRKWHKASNYIFVAIFWIVFLLLIVFYTAITKKIFKSHLKSSRNSTSVKKKSSRNIFSIVFVFFVCFVPYHIARIPYTKSQTEAHYSCQSKEILRYMKEFTLLLSAANVCLDPIIYFFLCQPFREILCKKLHIPLKAQNDLDISRIKRGNTTLESTDTL

SEQ ID NO: 10 - DNA, 1037 bp, Artificial Sequence
  Artificial sequence encodes chimpanzee P2RY14
ggtaccaccaccatgatcaattcaacctccacacagcctccagatgaatcctgctctcagaacctcctgatcactcagcagatcattcctgtgctgtactgtgtggtcttcattgcgggaatcctcctcaatggagtgtcaggatggatattcttttacgtgcccagctctaagagtttcattgtctatctcaagaacattgttattgctgactttgtgatgagcctgacttttcctttcaagatccttggtgactcaggccttggtccctggcagctgaacgtgtttgtgtgcagggtctctgccgtgctcttctacgtcaacatgtacgtcagcattgtgttctttgggctcatcagctttgatagatattataaaattgtaaagcctctttggacttctttcatccagtcagtgagttacagcaaactcctgtcagtgatagtatggatgctcatgctgctccttgctgttccaaatattattctcaccaaccagagtgttagggaggttacacaaataaaatgtatagaactgaaaagtgaactgggacggaagtggcacaaagcatcaaactacatcttcgtggccatcttctggattgtgtttcttttgttaatcgttttctatactgctatcacaaagaaaatctttaagtcccaccttaagtcaagtcggaattccacttcggtcaaaaagaaatctagccgcaacatattcagcattgtgttcgtgttttttatctgttttgtaccttaccatattgccagaatcccctacacaaagagtcagaccgaagctcattacagctgccagtcaaaagaaatcttgcggtatatgaaagaattcactctgctactatctgctgcaaatgtatgcttggaccctattatttatttctttctatgccagccatttagggaaatcttatgtaagaaattgcacattccattaaaagctcagaatgacctagacatttccagaatcaaaagaggaaatacaacacttgaaagcacagatactttgtgagcggccgc

SEQ ID NO: 11 - protein, 338 aa, Artificial Sequence; identical to SEQ ID NO: 6
  Chimpanzee P2RY14 receptor encoded by SEQ ID NO10

SEQ ID NO: 12 - DNA, 1832 bp, Artificial Sequence
  Artificial sequence encodes mouse P2RY14 receptor
ggatccatggaaggccgccaccccagcagactgaagccagacgtgaaggagttcatgtaagggagtccctgctgtcctccagacacactgatgcctgggctacggatggggacggggacgcaatgtgtctggaattctctcttccgaatcctggattctgttgacgaagcttgcctttgagattcctgaacacggagaaatagagattaaaaaccccagaagagagaaagtaaatgattcacaatcttgatgggttttgccgtatttatgttcttccactgtattagataccagtcacaaatgacttagaggccataaactgtgctttaagtaactagcctgcctttctatccagatctttgcctccagaggtgagaagatgaacaactccaccaccacagaccctccaaaccagccctgctcctggaacaccctgatcacaaagcagatcattcccgtgttgtacggtatggtcttcatcacggggctcctcctcaatgggatatcaggatggatattcttttatgtgcccagctccaagagttttatcatctatctcaagaacatagtggtggctgactttctcatgggcctgactttccctttcaaagtccttggtgactcaggcctcggcccctggcaggtgaatgtgtttgtgtgcagggtctctgccgtcatcttctatgttaatatgtacgtcagcatcgtgttctttgggctcatcagctttgacaggtactataaaattgtgaagccccttctgacgtctattgtgcagtcggtgaactatagcaagctgctttctgtgctcgtgtggatgctcatgcttctccttgctgtcccaaacatcatcctgacaaaccagggtgtcaaggaggtcacgaagatacagtgcatggagctcaaaaacgagctggggcggaagtggcacaaggcgtctaactatatcttcgtgagtatcttctgggtcgtgtttcttctgctaatcgtcttctacacggccatcacgaggaagatcttcaagtctcacctcaagtccaggaagaattccacctccgtcaagaggaagtccagccgcaatatcttcagcatcgtgctcgtttttgtcgtctgctttgtgccttaccacattgccagaatcccctacacaaagagtcagacggaaggtcactacagctgccggacgaaggagaccctgctctatgcgaaagaattcactctgctactctcggctgccaatgtgtgtctggaccccattatttatttcttcttatgccagccatttagagaagtcttaaataagaagttacacatgtcactcaaagtccagaatgacctagaggtttccaaaaccaaaagggaaaatgcgattcatgaaagcacagatactttgtaaattcccatccccttccaagtattatcagtcttgttacatgataattaagatacatgaataaaaagcaggcatatgatgataagtaacttagctagcaatatatctaataatatgtatgaagtccaaaaaggtataataaaaataaaatataagtttccatgcaaaatggaagtntgtagcacatcacatttttttagaaatcaaaggaacagagaagtggctttgtgggtgctggcgtatgagttaccaaaaccaaacttctcttctattaactggcttcttagaagacacccagtctttccgaccttcctcctaagcattcttccaagcaacactcgtatctatttcatgctttgtactatgcatgtgccaataaacaagttgtcttcaaaacccaaaaaaaaaaaaaaaaaaaaaaaagggcggccgcaagctt

SEQ ID NO: 13 - protein, 338 aa, Artificial Sequence; identical to SEQ ID NO: 5
  Mouse P2RY14 receptor encoded by SEQ ID NO12